(19) United States
(12) Patent Application Publication
(10) Pub. No.: US 2009/0202446 A1
(43) Pub. Date: Aug. 13, 2009

(54) MICROEMULSION COMPOSITION FOR AIR FRESHENER

(76) Inventors: Florin Joseph Vlad, Annandale, NJ (US); Nicholas O'Leary, Pennington, NJ (US)

Correspondence Address: WINSTON & STRAWN LLP;PATENT DEPARTMENT, 1700 K STREET, N.W., WASHINGTON, DC, 20006, US

(21) Appl. No.: 12/299,098

(22) PCT Filed: Apr. 19, 2007
(86) PCT No.: PCT/IB07/51410

§ 371 Date: Oct. 30, 2008

Related U.S. Application Data
(60) Provisional application No. 60/797,563, filed on May 3, 2006.

(30) Foreign Application Priority Data
Jul. 12, 2006 (EP) 06117017.1

(51) Int. Cl. A61K 9/12

(52) U.S. Cl. 424/45

(57) ABSTRACT

The present invention relates to air freshener devices of water-based liquid compositions, preferably clear liquids, that provide an improved diffusion of the composition and allow the use of fragrance materials with a wide range of characteristics, namely a wide range of clogP values. The compositions are clear microemulsions particularly adapted for use in automatic air fresheners and comprise at least 40% w/w of an aqueous phase, preferably water, an active oil in an amount of between 0.01 and 5% w/w, an oil-solubilizing system which is formed of a surfactant mixture of ionic and non-ionic surfactants, a solubilizing-aid ingredient in an amount of up to 5% w/w, and a propellant system in an amount of up to 40% w/w, wherein the perfume oil to total surfactant mixture is present at a weight ratio of between 0.2 and 4.5.

### TECHNICAL FIELD

The present invention relates to pressurized aerosol dispensers for volatile substances, namely fragrance-releasing devices, commonly designated as air freshener or deodorizer devices, intended for being manually or automatically activated to release perfuming or freshening compositions into the surroundings of the air freshener. They contain water-based liquid compositions, preferably clear liquids, that provide stable, clear, single-phase products with low level of volatile organic compounds (VOCs), and display excellent atomization characteristics, whilst allowing the use of fragrance materials with a wide range of characteristics, namely a wide range of ClogP values. The compositions of the invention are clear oil-in-water (o/w) microemulsions capable of carrying a perfume oil of any composition and concentration.

### BACKGROUND

Pressurized aerosol dispensers for fragrances or other volatile substances have been described in the prior art. Such devices are commonly used to refresh, deodorize, perfume or purify the air of rooms and enclosed spaces surrounding the fragrance-dispensing device or air freshener. Amongst these, the devices that can automatically spray the composition to be released, by means of regular or irregular bursts of the composition into the surrounding air, form an attractive class of products, providing periodic refreshing of rooms with a pleasant and/or purifying spray.

Automatic fragrance dispensers for freshening rooms and other closed spaces commonly comprise means capable of monitoring for example movement or light in the room and, as a result, initiate a fragrance dispensing event in response to the sensed parameter. This is described in a general manner for example in the recent US 2005/0171634 A1 publication, relating to the freshening of washrooms.

In this context, there can also be cited for example the device described in U.S. Pat. No. 6,644,507 B2, comprising a light sensor to which there is connected a circuit capable of controlling the discharge of purifying substance from a container into the room at a desired moment. Another representative example of this type of air fresheners is disclosed in EP 0956 868 B1, wherein the described device comprises two pressurized sources of fragrance so as to ensure that the latter is dispensed from a second canister when a first one becomes depleted. Many other examples can certainly be found in the literature.

The conventional fragrance or other dispensable formulations commercialized in this type of pressurized aerosols frequently suffer from several inconveniences. In many cases, they are dual phase systems requiring shaking prior to use, and this render the product unsuitable for automatic dispensing thereof. They commonly contain high levels of volatile organic compounds such as low alcohols, in order to provide single-phase compositions, and this is undesirable from an environmental point of view. Alternative, single-phase compositions resort to the use of fluorinated propellants and/or solvents to overcome the environmental concerns, but these tend to be high cost. Finally, it is not uncommon that their release of fragrance suffers from less than optimal, spray atomization characteristics when using formulations comprising solubilizers such as surfactants.

The present invention aims at solving all these problems by providing an air freshener composition which is a stable, clear, single-phase product, comprising low levels of VOCs, displays excellent atomization characteristics, is cost-effective and can be used with a wide variety of perfume substances. The aerosol formulations of the invention are thermodynamically stable, single-phase isotropic compositions under pressure in form of clear microemulsions.

### SUMMARY OF THE INVENTION

The present invention relates to a sprayable formulation capable of being contained in a pressurized vessel, in the form of a clear oil-in-water microemulsion, comprising at least 40% w/w of an aqueous phase, preferably water, an active oil in an amount comprised between 0.01 and 5% w/w, an oil-solubilizing system which is formed of a surfactant mixture and a solubilizing-aid ingredient, and a propellant system, wherein the propellant system is present in an amount of up to 40% w/w, the solubilizing-aid ingredient is present in an amount of up to 5% w/w, the surfactant system is a mixture of ionic and non-ionic surfactants and the weight ratio of perfume oil to total surfactant mixture is between 0.2 and 4.5, all w/w percentages being relative to the total weight of the formulation.

The invention further concerns a method of use of the above-mentioned composition in the form of an air freshener device to confer, improve, modify or enhance the odor of, and/or freshen, the air surrounding said composition and air freshener device, in rooms and open spaces, closets, cupboards and other closed environments.

### DETAILED DESCRIPTION OF THE INVENTION AND PREFERRED EMBODIMENTS

It is the object of the invention to provide a composition as recited above and a pressurized aerosol air freshener device for perfuming or freshening the surroundings thereof, comprising said composition.

The composition of the invention does not contain more than 40% w/w of volatile organic carbons (VOCs), such as a lower aliphatic alcohols having up to 5 carbon atoms as co-solvents, and/or dimethylether (DME) as a propellant. Preferably, the compositions are free of volatile organic co-solvents. By “VOC” we mean here the Volatile Organic Compounds as defined by the Environmental Protection Agency, and in particular we mean C₁-C₅ alkanols, such as ethanol, or C₁-C₅ alkanediols, such as ethylene glycol.

Throughout this description, the abbreviation w/w represents the weight-to-weight ratio between two ingredients, and particularly the weight of a specific ingredient relative to the total weight of the microemulsion.

The compositions of the invention are essentially water-based microemulsions, comprising an original oil-solubilizing system which is formed of at least one ionic and one non-ionic surfactant mixture, together with a solubilizing-aid ingredient, present in the proportions indicated above. The ionic surfactant component is preferably an anionic surfactant.

By “microemulsion” we mean here a dispersion that forms spontaneously and has a droplet size comprised between 10 and 150 nm, at a temperature comprised between 0° and 80° C. However, according to a particular embodiment of the invention, the present microemulsion has a droplet size comprised between 10 and 60 nm, or even between 10 and 40 nm, at a temperature comprised between 0° and 80° C. Such compositions are in fact also commonly designated as nanoemulsions or nanodispersions.

The compositions comprise at least 40% w/w of aqueous phase. The latter may be essentially formed of water or may also contain other hydrophilic ingredients having beneficial properties for the microemulsion or its use, such as viscosity modifiers, colorants, preservatives, humectants, etc. Preferably, the aqueous phase will form at least 60% w/w of the microemulsion formulation, and it may even go up to 80% w/w of the latter.

In some cases, it may be beneficial to include a co-solvent, namely a lower aliphatic alcohol such as ethanol, but never above 30% w/w relative to the microemulsion weight.

The microemulsions of the invention carry oil, more particularly a hydrophobic fragrance composition. These are typically lipophilic organic liquids essentially insoluble in water. By a “perfume” or a “fragrance”, as used throughout this text, it is meant here an ingredient, or a mixture of ingredients, which are primarily intended to impart an odor to, and/or freshen the, surroundings of the air-freshener device.

This is typically a lipophilic organic liquid that is essentially insoluble in water. An example of a suitable oil to be solubilized is a liquid that comprises at least 75% w/w, or even at least 90% w/w, of a perfume or a perfuming composition.

In particular, as the perfume or perfuming composition there can be used any perfuming ingredient or, as happens more often, any mixture of perfuming ingredients currently used in perfumery, e.g. of compounds capable of imparting pleasant odor to the air or closet surroundings of the air-freshener. Said perfuming ingredients can be of natural or synthetic origin. A detailed description of said ingredients would not be warranted here and, in any case, it cannot be exhaustive. Generally speaking, it can be mentioned that these ingredients belong to chemical classes as varied as alcohols, aldehydes, ketones, esters, ethers, acetates, nitriles, terpenic hydrocarbons, nitrogenous or sulphurous heterocyclic compounds and essential oils of natural or synthetic origin. The nature of these ingredients can be found in specialized books of perfumery, e.g. in S. Arctander (Perfume and Flavor Chemicals, Montclair N.J., USA 1969 or later versions thereof), or similar textbooks of reference. The selection of such ingredients is carried out by the perfumer without particular difficulty, on the basis of her/his general knowledge and as a function of the desired sensory effect, i.e. the perfuming effect that is to be imparted to environing surroundings.

The nature of the perfume ingredients is totally immaterial for the intended effect of the compositions and devices of the invention. As a result of the presence of the solubilizing-aid ingredient or ingredients in the liquid composition of the invention, it is possible to solubilize any perfume ingredient, or mixture of ingredients, regardless of the vapor pressure or ClogP values thereof.

Therefore, it is possible to use, as the solubilized oil, low polarity oils, in particular a low polarity perfume. By “low polarity oil or perfume” we mean here, for example, an oil or perfume rich in highly hydrophobic ingredients or an oil or perfume that contains only small amounts of polar solvents, or yet is completely free of polar solvents.

As low polar perfumes one can mention those containing from 5% w/w, or even 20% w/w, to 99% w/w of terpenes and/or from 5 to 30% w/w of musks; percentages being relative to the weight of the solubilized perfume oil.

Said terpenes may be of wood or citrus origin and example of which are terpineol. or d-limonene. A non-restrictive example of musks is hexadecanolide or Habanolide® (15-pentadec-11,12-enolide; origin: Firmenich S A, Geneva, Switzerland) and similar macrolides of current use in perfumery. This type of ingredients is particularly hydrophobic and known to be highly difficult to solubilize in aqueous based compositions to provide clear products.

As mentioned above, the perfume oil typically represents 0.01 to 5% w/w of the liquid nanodispersion total weight.

The perfume may also contain a suitable solvent, in a quantity of up to 25% w/w of the perfume oil, but preferably less than 10% w/w. The presence of a solvent may be useful to obtain a monophasic oil or to modulate the surface tension of said oil. As examples of suitable solvents, one may cite polar or non-polar low molecular weight solvents such as isoparaffins, paraffins, hydrocarbons, silicon oils, perfluorinated aliphatic ethers, glycol ethers, glycol ether esters, esters, or ketones. Non-restrictive examples of such solvents include dimethicone or cyclomethicone, which are commercialized by Chemsil Silicon INC. under the trade names Cosmetic Fluid® 1288, and respectively Cosmetic Fluid® 1387, jojoba oil, perfluoroisobutyl methyl ether, diethyl phthalate, dipropylene glycol and isopropyl myristate.

The air-freshening compositions of the invention further comprise an oil solubilizing system formed of a surfactant mixture and a solubilizing-aid ingredient. The use of a suitable amount of an appropriate solubilizing-aid ingredient, in addition to the classical ingredients of the aqueous air freshener compositions such as the perfume, the surfactant system and water, makes it possible to modulate at will the nature of the fragrance used, for a defined surfactant system.

The surfactant system is preferably a mixture of a primary ionic surfactant with a secondary non-ionic surfactant. The ionic surfactant is preferably an anionic surface active substance.

Suitable anionic surfactants comprise the salts of C₆-C₂₄ mono- or di-sulfonic, alkylsulfuric, alkylarylsulfuric, alkylarylphosphate or carboxylic acids and also the polyethylene glycol co-polymers with sulfonic or carboxylic acids. Specific, but not limiting examples of said anionic surfactants are sodium, potassium, ammonium or mono-, di- or tri-ethanolammonium salts of C₆-C₁₂ dialkyl sulfosuccinic acids (such as sodium dioctyl-sulfosuccinate, namely the products commercialized by Cytec Industries, Inc. under the tradename Aerosol OT, in the form of flakes or solutions), C₇-C₂₄ alkarylsulfonic acids (such as sodium dodecyl benzenesulfonate), C₆-C₁₅ alkylsulfuric acid (such as sodium dodecylsulfate), C₁₀-C₂₀ acyl glutamic acid (such as sodium cocoyl glutamate), or polyethylene glycol/dimethicone sulfosuccinic acids (such as disodium PEG-12 dimethicone sulfosuccinate known under the trade name Mackanate® DC-50 from The McIntyre Group).

Suitable cationic surfactants comprise the salts of C₁₀-C₃₅ ammonium derivatives of fatty acids, alcohols, alkylamidoalkylmorpholine or amines and also the IPDI (isophorone diisocyanate) co-polymers with said ammonium derivatives or with fatty amines and optionally polyethylene glycols. Specific, but not-limiting examples, of suitable cationic surfactants are halides, sulfates or carboxylates of C₂₀₋₃₀ quaternary ammonium alkyls (such as hexadecyltrimethyl ammonium bromide or didodecylammonium bromide), C₁₋₄ alkyl N-cocoyl-L-arginate (such as DL-2-pyrrolidone-5-carboxylic acid salt of ethyl N-cocoyl-L-arginate commercialized by Ajinomoto Co., Inc. under the trade name CAE®), (C₁₀₋₂₀ amido) (C₁₋₁₄ alkyl) morpholine (such as isostearamidopropyl morpholine lactate), IPDI copolymers with N—C₁₀₋₂₀ amido(C₁₋₄ alkyl)-N,N-di(C₁₋₄ alkyl)-N—(C₁₋₄ alkyl) Ammonium (such as bis(N-Ricinolemidopropyl-N,N-Dimethyl)/N-Ethyl Ammonium Sulfate/IPDI Copolymer also known under the trademark Polyquat® PPI-RC from ALZO) or polyethylene glycol/C₁₀-C₂₀ fatty alkyl amine/IPDI copolymers (such as the PEG Cocamine/IPDI Copolymeric surfactants also known under the trademark Polyderm® PPI-CA-15 from ALZO).

Amphoteric surfactants can also be used and suitable ones comprise C₁₀-C₂₅ betaines, amphoacetates and imidazoline derivatives, as well as the polyethylene glycol/fatty amine/glycine/IPDI copolymers. Specific, but non limiting, examples of said amphoteric surfactants are the C₁₀-C₂₀ fatty amido C₂-C₅ alkyl betaines (such as cocoamidopropyl betaine), coco- and lauro-amphoacetates (such as sodium cocoamphoacetate known under the trade name Mackam® HPC-32 commercialized by McIntyre Group), and the polyethylene glycol/C₁₀-C₂₀ fatty alkyl amine/glycine/IPDI copolymers (such as PEG-13 soyamine-Glycine/IPDI Copolymer also known under the trademark Polytaine® PPI-SA-15 from ALZO).

The non-ionic secondary surfactants include ethoxylated and/or propoxylated (C₅-C₁₂ alkyl)phenols ethers containing 5 to 20 EO or PO units (such as polyethylene glycol nonylphenyl ethers, polyethylene glycol octylphenyl ethers, also known under the generic tradename Polystep®), polyethylene glycol sorbitol ether containing 3 to 30 EO units (such as sorbitol esters with oleic, myristic, stearic, palmitic acid also known as those known under the tradenames Tween® from ICI or Glycosperse® from LONZA), sucrose esters with C₈-C₂₀ fatty acid (such as sucrose esters with oleic, palmitic or stearic acid, such as Ryoto Sugar Ester M-1695 commercialized by Mitsubishi-Kagaku Foods Corporation), ethoxylated aliphatic C₆-C₂₀ alcohols containing 2 to 30 EO units (such as ethoxylated secondary C₆-C₂₀ alcohols), C₈-C₂₀ polyglyceryl esters (such as glycerol-polyethylene glycol oxystearate commercialized by BASF under the trade name Chremophor® CO40), polyethylene glycol and polypropylene glycol block copolymers (such as those known under the tradename Pluronics® from BASF), ethoxylated glycol ether containing 2 to 30 EO units (such as PEG-10 stearyl ether also known under the trade name Volpo® S-10 from CRODA), or polyethylene glycol mono- or -diester of aliphatic C₅-C₁₁ carboxylic acids containing 2 to 10 EO units (EO stands for ethylene oxide and PO stands for propylene oxide). Useful and advantageous examples are the (polyoxyethylene)-nonylphenylethers with HLB between 6 and 18, such as MAKON®-10 from STEPAN Company, or Secondary (Ethoxylated) Alcohols with HLB between 6 and 18, such as TERGITOL®15-S-9 from The Dow Chemical Company or any other non-ionic surfactants with the HLB value between 6 and 18, such as the copolymeric Pluronic® F-68 surfactants from BASF. According to specific embodiments of the invention there will be used non-ionic surfactants having an HLB below 13.

In the embodiments of the invention, the weight ratio of surfactant system versus perfume oil is preferably kept within the range between 0.2 and 4.5, more preferably in a range comprised between 0.5 and 3.0.

The oil-solubilizing system of the invention further comprises a solubilizing-aid ingredient. According to a preferred embodiment, this ingredient is selected from the group consisting of ethanol, dimethyl ether (DME) or diethyl ether (DEE) and acetone. The amount of ethanol used shall not be above 30% w/w of the microemulsion.

According to alternative embodiments, the solubilizing-aid ingredient further contains at least one organic or inorganic salt, or a precursor thereof, of low molecular mass, e.g. below 400 g/mol. Suitable such salts include the compounds cited on page 3 and in the examples of US 20040209795 A1 belonging to the same applicant, the contents of which are hereby included by reference. Preferably, these salts are selected from the group consisting of sodium, potassium, magnesium and calcium salts of pyridine carboxylic acids, proline acid, pyrrolidone carboxylic acid, benzoic acid, hydroxyl-benzoic acid, amino-benzoic acid, lactic acid, ascorbic acid, bicarbonate, succinic acid, oxalic acid, tartaric acid, citric acid, a C₈-C₁₀ derivative of benzoic, hydroxyl-benzoic or amino-benzoic acid substituted by one or two C₁-C₃ alkyl groups (such as the sodium salt of p-methyl-benzoic acid or of p-isopropyl-hydroxyl-benzoic acid), benzene-sulfonic acid, a C₇-C₉ benzene-sulfonic acid substituted by one or two methyl or ethyl groups (such as potassium toluene sulfonate), optionally hydroxylated naphthalene-sulfonic acid, an optionally hydroxylated C₁₁-C₁₆ naphthalene-sulfonic acid substituted by one or two C₁-C₅ alkyl groups (such as sodium butylnaphtalene sulfonate), C₃ to C₆ alkanoic acids (such as the sodium salt of pentanoic acid), and any mixture of said salts.

Most preferably, these compounds are advantageously chosen amongst the following compounds: pyrrolidone carboxylic acid sodium salt (also known as Ajidew® NL-50 from Ajinomoto), sodium benzoate, sodium molybdate, sodium borate, sodium L-lactate, calcium L-ascorbate, sodium bicarbonate and di-sodium succinate, and mixtures thereof. Sodium benzoate and sodium lactate are particularly useful salts for the purposes of the invention.

As mentioned above, the solubilizing-aid ingredient is present in an amount such that the microemulsions according to the invention are clear and which is of up to 5% w/w, relative to the total weight of microemulsion.

In each particular microemulsion sample, the amount of solubilizing-aid ingredient to obtain a clear product according to the invention depends on the exact nature of the oil, on the surfactant mixture, and on the amount of perfume oil present. The person skilled in the art is able to thus adjust the necessary amount of solubilizing-aid to obtain the desired clear microemulsions, provided that the relative concentration ranges defined above are observed.

The microemulsions carrying a fragrance also contain a propellant. The latter is present in the compositions of the invention in an amount of up to 40% w/w of the entire formula. More preferably, its concentration shall represent between 25 and 35% w/w of the whole formulation, and best modes of execution comprise 20 to 30% w/w of propellant.

According to advantageous embodiment of the invention, the propellant system comprises the solubilizing-aid ingredient and is selected from the group consisting of ethanol, dimethyl ether (DME) or diethyl ether (DEE), acetone and mixtures thereof. The microemulsions of the invention can thus be pressurized and used in the air freshener devices according to the invention. The use of dimethyl ether (DME) as both the solubilizing-aid and propellant ingredients is particularly advantageous, possibly in admixture with ethanol, the content of the latter being less than or equal to 10% w/w of the total microemulsion formulation.

The above liquid compositions are clear, water-based, fragrance carriers intended to be diffused in closed spaces such as cupboards, drawers and closets, or into the surrounding environment of air freshener devices, such as open spaces and rooms. They are particularly adapted for use in pressurized aerosol-type air freshener devices that do not suffer from the inconveniences associated with prior known devices of the same type.

By a “composition for an air freshener device” it is meant here a composition that is in a form appropriate to be diffused into its surroundings via an air freshener device of this type. The composition may therefore also comprise optional ingredients such as corrosion inhibitors, anti-oxidants, dyestuffs, bittering agents, UV inhibitors, preservatives, chelating agents and any other appropriate oil or water soluble ingredients, current in this type of device.

Whenever said ingredients are added to the clear liquid air-freshening compositions, then they will represent no more than 3% w/w, or even 2% w/w, the percentages being relative to the total weight of the composition.

Non-limiting examples of suitable corrosion inhibitors for the present invention include sodium molybdate, sodium borate and sodium nitrite.

Water soluble dyestuffs suitable for the present invention can be found in the Colour Index International published by The Society of Dyers and Colourists. The inclusion levels of suitable dyestuffs is typically between 0.005 to 0.5% w/w. Non-limiting examples of dyes suitable for the present invention include: Vitasyn Quinoline Yellow 70, C.I. No. 47005; Vitasyn Tartrazine X 90, C.I. No. 19140; Sanolin Yellow BG, C.I. No. 19555; Vitasyn Orange RGL 90, C.I. No. 15985; Vitasyn Ponceau 4RC 82, C.I. No. 16255; Sanolin Red NBG, C.I. No. 18134; Sanolin Rhodamin B, C.I. No. 45100; Sanolin Violet E2R; Sanolin Violet FBL; Duasyn Ink Blue SLK, C.I. No. 42780; Nylosan Blue PRL 200, C.I. No. 62058; Sanolin Blue NBL, C.I. No. 61585; Duasyn Acid Blue AE 02, C.I. No. 42090; Vitasyn Blue AE 90, C.I. No. 42090; Sanolin Green R-3GL; Sanolin Green 6GLN. All dyestuffs origin: Clariant.

Non-limiting examples of other water soluble ingredients that may be added to the composition include one or more of the following: ethanol; isopropanol; ethylene glycol; propylene glycol; diethylene glycol; dipropylene glycol; propylene glycol monomethyl ether; dipropylene glycol monomethyl ether; tripropyleneglycol monomethyl ether; propylene glycol n-butyl ether; dipropylene glycol n-butyl ether; tripropylene glycol n-butyl ether, propylene glycol n-propyl ether; dipropylene glycol n-propyl ether; Eastman EP (origin: Eastman Chemical Company); Eastman EB (origin: Eastman Chemical Company); Eastman EEH (origin: Eastman Chemical Company); Eastman DM (origin: Eastman Chemical Company); Eastman DE (origin: Eastman Chemical Company); Eastman DP (origin: Eastman Chemical Company); Eastman DB (origin: Eastman Chemical Company).

Particularly useful air freshening compositions of the invention comprise between 1.5 and 7% w/w of surfactant system, around 2% w/w of perfume oil, around 30% w/w of dimethyl ether or a mixture thereof with ethanol, the latter being present in an amount not above 10% w/w, the remaining being water.

The invention's compositions can be prepared according to any method known in the art. A suitable method consists in dissolving into the water the surfactant or perfume solubilizing system, to form a clear micellar solution which acts as a pre-microemulsion. Possibly, during this stirring stage a nitrogen blanket may be useful to avoid foaming and to protect the system from oxygen contamination. To the resulting clear micellar solution is added under gentle stirring the fragrance oil such that a milky emulsion results. Finally the latter is turned into an isotropic clear, single-phase product by addition of the solubilizing-aid ingredient, and whenever necessary the optional ingredients to form a clear dispersion.

According to another suitable method, one can proceed by dissolving into the water the surfactant system, to form a clear micellar solution. To the resulting micellar solution are added under gentle stirring the solubilizing-aid ingredient, and whenever necessary the optional ingredients to form an initial oil-free microemulsion. Under gentle mixing the resulting oil-free microemulsion can easily solubilize the corresponding amount of oil, namely the perfume, to form an isotropic clear, single-phase product. High mechanical forces such as shear forces are not necessary to manufacture the present air freshener compositions.

Usually a clear product is obtained in a short time, less than 30 min stirring, and sometimes even instantly.

The perfume compositions of the invention are non-toxic and safe for use, as well as cost-effective, thus rendering the air freshener devices particularly attractive and advantageous over prior known devices.

In addition to the perfuming ingredients as defined above, the perfume compositions of the invention may also contain other common air deodorizing, antibacterial or freshening ingredients, if it is desired to deodorize or purify the ambient air.

The liquid composition of the invention, when incorporated in an air freshening device, allows an improved performance thereof, particularly relative to prior known automatically activated aerosol devices.

The invention thus also provides an air freshener device comprising a container for carrying the liquid fragrance composition such as above-mentioned, and means for diffusing said composition into the surroundings of the air freshener upon activation of the air freshener device by a user. When necessary, e.g. during storage of the non-activated air freshener device, the latter also comprises means for preventing evaporation of the fragrance composition prior to activation of the air-freshener by the user, which means are removable to allow activation of the device when desired.

The nature and type of the constituents of such consumer products do not warrant a more detailed description here, which in any case would not be exhaustive, the skilled person being able to select them on the basis of its general knowledge and according to the nature and the desired effect of said product. It is clear that air fresheners according to the invention can take the form of those described in the prior art of reference cited in the introduction of this description and relating to pressurized air freshener devices, possibly activated automatically.

A further object of the present invention is the use of the air freshener of the invention to confer, enhance, improve, modify or freshen the odor and ambient air quality of a room, an open space, a closet or other closed space, via activation therein of the device to expose its surroundings to the perfume diffused.

The invention also relates to the use of the above-described liquid compositions to confer, enhance, improve, modify or freshen the odor and ambient air quality of a room, an open space, closet or other closed space.

### EXAMPLES

The following examples are further illustrative of the present invention embodiments.

### Examples 1 to 3

### Preparation of a Liquid Composition According to the Invention for Use in a Pressurized Air Freshener

Compositions according to the invention were prepared with the ingredients indicated in the Table 1, used in the proportions disclosed.

TABLE 1
Formulations according to the invention

| | Composition | | |
|---|---|---|---|
| | A | B | C |
| Ingredient | weight % | weight % | weight % |
| Aerosol OT-100 ¹⁾ | 0.79 | 2.79 | 3.36 |
| Glycosperse ® O-20 ²⁾ | 0.30 | 1.07 | 1.3 |
| Glycosperse ® L-20 ³⁾ | 0.04 | 0.12 | 0.15 |
| Makon ® 10 ⁵⁾ | 2.25 | — | — |
| Solubilisant LRI ⁴⁾ | 0.47 | 1.66 | 2.01 |
| Perfume | 2.00 | 2.00 | 2.00 |
| Ethanol | 10.00 | — | — |
| Water | 64.11 | 62.32 | 61.14 |
| DME | 20.00 | 30.00 | 30.00 |
| Sodium Molybdate | 0.007 | 0.007 | 0.007 |
| Sodium Borate | 0.034 | 0.034 | 0.034 |
| Total | 100.00 | 100.00 | 100.00 |

¹⁾ Dioctyl sulfosuccinate sodium salt; origin: Cytec Industries, Inc.
²⁾ Monooleate ethoxylated sorbitol with 20 EO; origin: Lonza Inc.
³⁾ Monolaureate ethoxylated sorbitol with 20 EO; origin: Lonza Inc.
⁴⁾ PPG-26 Buteth-26 & PEG-40 Hydrogenated Castor Oil & Water; origin: LWR Inc.
⁵⁾ Nonylphenol ethoxylated, POE-10; Origin: Stepan Company

The compositions were then introduced under pressure into canister or glass vessels.

1.-15. (canceled)

16. A composition intended for being dispensed via a pressurized air freshener for perfuming or freshening the surroundings thereof, which comprises a microemulsion, containing at least 40% w/w of an aqueous phase, an active oil in an amount comprised between 0.01 and 5% w/w, an oil-solubilizing system which is formed of a surfactant mixture and a solubilizing-aid ingredient, and a propellant agent, wherein:
a) the propellant agent is present in an amount of up to 40% w/w;
b) the solubilizing-aid ingredient is present in an amount of up to 5% w/w;
c) the surfactant system is a mixture of ionic and non-ionic surfactants; and
d) the weight ratio of perfume oil to total surfactant mixture is between 0.2 and 4.5;
all w/w percentages being relative to the total weight of the formulation.

17. The composition according to claim 16, comprising at least 60% w/w of water.

18. The composition according to claim 16, wherein the ratio of perfume oil to total surfactant mixture is between 0.5 and 3.

19. The composition according to claim 16, comprising a mixture of anionic and non-ionic surfactant or surfactants.

20. The composition according to claim 19, wherein:
a) the anionic surfactant is selected from the group consisting of sodium, potassium, ammonium and mono-, di- and tri-ethanolammonium salts of C₆-C₁₂ dialkyl sulfosuccinic acids, C₇-C₂₄ alkarylsulfonic acids, C₆-C₁₅ alkylsulfuric acid, C₁₀-C₂₀ acyl glutamic acid, and polyethylene glycol/dimethicone sulfosuccinic acids; and mixtures thereof; and
b) the non-ionic surfactant is selected from the group consisting of ethoxylated and propoxylated (C₅-C₁₂ alkyl)phenol ethers containing 5 to 20 EO or PO units, polyethylene glycol sorbitol ether containing 3 to 30 EO units, sucrose esters with C₈-C₂₀ fatty acid, ethoxylated aliphatic C₆-C₂₀ alcohols containing 2 to 30 EO units, C₈-C₂₀ polyglyceryl esters, polyethylene glycol and polypropylene glycol block copolymers, ethoxylated glycol ether containing 2 to 30 EO units, and polyethylene glycol mono- and -diester of aliphatic C₅-C₁₁ carboxylic acids containing 2 to 10 EO units, and mixtures thereof.

21. The composition according to claim 20, wherein the solubilizing-aid ingredient is selected from the group consisting of ethanol, dimethyl ether (DME) or diethyl ether (DEE), acetone, and mixtures of two or more of these, and wherein the amount of ethanol is comprised between 0 and 10% w/w, relative to the total weight of composition.

22. The composition according to claim 20, wherein the solubilizing-aid ingredient further comprises a salt selected from the group consisting of pyrrolidone carboxylic acid sodium salt, sodium benzoate, sodium L-lactate, sodium molybdate, sodium borate, calcium L-ascorbate, sodium bicarbonate, di-sodium succinate and any mixture of said salts.

23. The composition according to claim 20, wherein the solubilizing-aid ingredient also forms the propellant.

24. The composition according to claim 16, wherein the propellant is selected from the group consisting of ethers, alcohols, fluorinated alcohols, volatile hydrocarbons, nitrogen, argon, liquefied carbon dioxide and mixtures of one or more of these compounds.

25. The composition according to claim 24, wherein the propellant is DME or a mixture thereof with ethanol.

26. The composition according to claim 16, comprising between 1.5 and 7% w/w of surfactant mixture, around 2% w/w of perfume oil, around 30% w/w of dimethyl ether or a mixture thereof with ethanol, the latter being present in an amount not above 10% w/w, the remaining being water.

27. A pressurized air freshener device for perfuming or freshening the surroundings thereof, comprising a composition according to claim 1, and means for diffusing the composition into the surroundings.

28. The air freshener device according to claim 27, which is capable of being automatically activated.

29. A method to confer, enhance, improve, modify or freshen the odor and ambient air quality of a room, an open space, a closet or other closed space, which consists in activating therein an air freshener device according to claim 27, to expose its surroundings to the perfume composition contained in the activated air freshener.

30. A method to confer, enhance, improve, modify or freshen the odor and ambient air quality of a room, an open space, a closet or other closed space, which consists in activating therein a composition according to claim 16, to expose its surroundings to the perfume composition contained therein.
